(19) United States
(12) Plant Patent Application Publication
(10) Pub. No.: US 2019/0104660 P1
(43) Pub. Date: Apr. 4, 2019

(54) Citrus Ichangensis X C. Maxima Tree Named 'TIFT2-16'

(50) ;

(71) Applicant: University of Georgia Research Foundation, Inc., Athens, GA (US)

(72) Inventors: Wayne W. Hanna, Chula, GA (US); Brian M. Schwartz, Tifton, GA (US)

(73) Assignee: University of Georgia Research Foundation, Inc., Athens, GA (US)

(21) Appl. No.: 15/732,210

(22) Filed: Oct. 3, 2017

(51) Int. Cl. A01H 6/78

(52) U.S. Cl. PLT/201

(57) ABSTRACT

A new variety of Citrus ichangensis x C. maxima plant named ‘Tift2-16’ produces fruit with a reduced number of seeds, making it valuable as a backyard fruit tree. Also, it is a desirable tree for landscaping.

Latin name of the genus and species of the plant claimed: ‘Tift2-16’ is a vegetatively propagated Citrus cultivar of the genus and species Citrus ichangensis x C. maxima.

Variety denomination: The new tree is of the cultivar denominated ‘Tift2-16’.

### BACKGROUND OF THE INVENTION

The present invention relates to a new and distinct cultivar of Citrus ichangensis x C. maxima herein referred to as ‘Tift2-16’.

The new Citrus ichangensis x C. maxima is a product of a planned research, evaluation, and testing program conducted by the Inventors in Tifton, Ga. The objective of the Citrus ichangensis x C. maxima research program is to create a new plant cultivar with reduced seed production. This cultivar is commercially important for its low seed production. These and other qualities are enumerated herein.

Pedigree and history: ‘Ichang’ lemon (not patented) is an old Chinese hybrid cultivar that has fruit that can be used as a lemon. It has large juicy fruit and has been grown by backyard Citrus growers for more than 75 years across the southern half the Coastal Plain in the United States. It will reliably grow without protection up to the northern border of US Hardiness Zone 8b. It has been reported that ‘Ichang’ will be hardy to 12° C. It will grow further north with protection. ‘Ichang’ produces a juicy fruit with many large seeds (the main complaint about this cultivar). Years ago before all of the quarantine regulations, gardeners from Louisiana to North Carolina brought ‘Ichang’ fruit yearly to the Southeastern Citrus Expo (a part of the Southeastern Palm Society). Our objective on this project was to reduce the number of seed in each fruit in hopes that this cultivar would be more widely used in backyard gardening.

On Jan. 14, 2003, we irradiated seeds of ‘Ichang’ lemon harvested from a tree (established from seed) that had been growing in one of the inventor's yards since 1976. These seeds were removed from the fruit and immediately irradiated with 25 Kr of gamma radiation. The seeds were planted in large pots in a greenhouse and individual seedlings (when about 8 cm tall) were transplanted to 10 cm pots. In June 2003, 125 seedlings produced from the irradiated seeds were transplanted to a test field on 3.05 m center to center spacing. These trees began producing fruit in 2009. In 2009, we began cutting fruit systematically from around each tree, looking for seedless chimeras. We identified one of these trees, which we have called ‘Tift2-16’, in December 2009 as having the best fruit and lowest seed set and began evaluating its fruit characteristics. We began grafting and budding trees from this original ‘Tift2-16’ tree in January 2010 in Tifton, Ga. onto Poncirus trifoliata (non-patented) rootstock. We planted these asexually propagated trees in the field in April 2010. Scion wood was taken from various branches of the original tree of this new variety and used to propagate trees of the new variety. Observations of fruit from these trees has confirmed that these trees reproduce true for the reduced seed characteristic.

Asexual reproduction of the new Citrus ichangensis x C. maxima ‘Tift2-16’ by vegetative propagation (budding and grafting) in La Grange, Ga.; Pavo, Ga., Tifton, Ga., and Valdosta, Ga. in 2013, 2014, and 2015, has shown that the unique features of this new Citrus cultivar are stable and reproduce true to type in successive generations.

### SUMMARY OF THE INVENTION

The following traits have been repeatedly observed and represent the characteristics of the new cultivar ‘Tift2-16’. The new cultivar ‘Tift2-16’ has not been observed under all possible environmental conditions. We have not tested ‘Tift2-16’ outside of the state of Georgia because of APHIS regulations. The phenotype may vary somewhat with variations in, for example, temperature, day-length, light intensity, soil types, water and fertility levels without, however, any variance in genotype.

The following traits have been repeatedly observed for the new C. ichangensis x C. maxima cultivar ‘Tift2-16’ in Pavo, Ga., Tifton, Ga., and Valdosta, Ga.

1. ‘Tift2-16’ exhibits a vigorous growth habit.

2. ‘Tift2-16 has a significantly reduced number of seeds per fruit.

The new cultivar Citrus ‘Tift2-16’ can be compared to its parent, Citrus ichangensis x C. maxima var. Ichang.

Plants of the new Citrus “Tift2-16’ differ from its parent ‘Ichang’ for plants growing at Pavo, Ga.; Tifton, Ga.; and Valdosta, Ga. in that ‘Tift2-16’ produces significantly fewer seeds per fruit compared to ‘Ichang’.

In addition, observations of ‘Tift2-16’ and ‘Ichang’ plants of the same age and growing on the same rootstock variety in Tifton, Ga. confirmed: (i) ‘Tift2-16’ leaflet blades are wider than those of ‘Ichang’; (ii) ‘Tift2-16’ petioles have a shorter wing length than those of ‘Ichang’; (iii) ‘Tift2-16’ petioles are longer than those of ‘Ichang’; and (iv) ‘Tift2-16’ juice has a lower pH than ‘Ichang’ juice.

The following observations, measurements, and values describe plants grown in Pavo, Ga.; Tifton, Ga.; and Valdosta, Ga. In Tables 1 and 2, the least significant difference (LSD) is set at P≤0.05 probability level. Trees were spaced 3.05 meters on centers. Trees were grafted to Poncirus trifoliata in a greenhouse in January and transplanted to the field in mid-April. Fruit and tree characteristics were rated in November.

We evaluated Tift2-16, a reduced seed chimera, from 2011 thru 2015 in the following Georgia locations in various years: Tifton, Ga.; Valdosta, Ga.; LaGrange, Ga.; and Pavo, Ga. ‘Tift2-16’ did survive north of LaGrange from 2011 thru 2013, but did not survive the −11° C. to −12° C. temperatures that occurred for two full days during January 2014. ‘Tift2-16’ performed well from 2013 to 2015 without protection at Tifton, Ga; Valdosta, Ga; and Pavo, Ga. No temperatures dropped below −7° C. (20° F.) from 2004 thru 2016 at Tifton, Ga. according to personal and historical records. The coldest temperature at Tifton, Ga. since 2003 was −10° C. on Jan. 24, 2003.

‘Tift2-16’ produced significantly fewer seeds per fruit than ‘Ichang’ on all trees tested (Table 1). Seed set on the ‘Tift2-16’ chimera in 2012 was 6.5 per fruit compared to 50 seeds per fruit for ‘Ichang’ (LSD—5%=9.4, data not included in Table 1). The fruit of ‘Tift2-16’ was observed to be approximately equal, in many examples, or significantly smaller in both circumference and fruit weight compared to ‘Ichang’ fruit. This was somewhat expected because ‘Ichang’ has many large seeds so consequently, fewer seeds resulted in smaller fruit. Juice of ‘Tift2-16’ tended to have a Brix value equal to or significantly lower than ‘Ichang’. Most fruit from ‘Tift2-16’ produced a juice volume similar to the juice volume from ‘Ichang’ fruit. Juice pH was 2.3 and 2.5 for ‘Tift2-16 and ‘Ichang’, respectively (LSD—5%=0.1) in November 2015.

In summary, ‘Tift2-16’ is similar to ‘Ichang’ in fruit and leaf characteristics except that ‘Tift2-16’ produces significantly fewer seeds than ‘Ichang’; which should greatly increase the desirability of this species as a backyard fruit.

TABLE 1
Fruit characteristics^{z} of fruit from several ‘Tift2-16’ trees and from one ‘Ichang’ tree for
fruit harvested in 2013, 2011 and 2015.

| Tree | | | Fruit^{y} | | | | | |
|---|---|---|---|---|---|---|---|---|
| Tree | Age | | Circum. | Weight | | Juice | Seed | Number |
| Number^{x} | Years | Location^{w} | (cm) | (g) | Brix | (ml) | No. | Per Tree |
| ‘Tift2-16’ | | | 2013 | | | | | |
| 3 | 3 | Tifton | 25.0 | 239 | 8.7 | 111 | 8 | NR |
| 4 | 3 | Tifton | 23.7 | 189 | 12.2 | 75 | 6 | NR |
| ‘Ichang’ | >30 | Chula | 30.0 | 380 | NR | NR | 56 | NR |
| LSD-5% | | | 0.8 | 62 | 1.6 | 38 | 12 | |
| ‘Tift2-16’ | | | 2014 | | | | | |
| 3 | 4 | Tifton | 24 | 216 | 9.5 | 136 | 1 | NR |
| 7 | 4 | Tifton | 30 | 345 | 8.3 | 150 | 5 | NR |
| 8 | 4 | Tifton | 25 | 179 | 7.6 | 94 | 5 | NR |
| 10 | 4 | Valdosta | 29 | 322 | 7.5 | 146 | 5 | NR |
| 11 | 4 | Pavo | 28 | 314 | 8.7 | 152 | 7 | NR |
| 15 | 3 | Tifton | 25 | 240 | 8.2 | 119 | 6 | NR |
| ‘Ichang’ | >30 | Chula | 32 | 479 | 8.7 | 162 | 56 | NR |
| LSD-5% | | | 4.5 | 61 | 0.4 | 32 | 5.9 | |
| ‘Tift2-16’ | | | 2015 | | | | | |
| 3 | 5 | Tifton | 28 | 248 | 9.5 | 144 | 7.5 | 84 |
| 5 | 5 | Tifton | 27 | 306 | 7.5 | 149 | 7.2 | 44 |
| 6 | 5 | Tifton | 29 | 327 | 8.4 | 153 | 7.0 | 56 |
| 7 | 5 | Tifton | 27 | 296 | 8.8 | 148 | 7.7 | 104 |
| 9 | 4 | Tifton | 29 | 372 | 7.6 | 166 | 5.5 | 15 |
| 12 | 4 | Tifton | 27 | 316 | 7.6 | 138 | 6.0 | 11 |
| 13 | 4 | Tifton | 28 | 294 | 8.2 | 145 | 6.5 | 49 |
| 14 | 3 | Tifton | 29 | 304 | 8.2 | 146 | 6.2 | 12 |
| ‘Ichang’ | >30 | Chula | 29 | 343 | 8.6 | 126 | 56 | 100 s |
| LSD-5% | | | 2 | 52 | 0.7 | 31 | 7.1 | |

Data were taken on 21 Nov. 2013, 10 Nov. 2014, and 20 Nov. 2015. Each tree mean is from five random fruit from a tree. Number Per Tree is the actual or estimated number of fruit per tree.
‘Ichang’ is the original lemon tree at Chula, Ga. that served as the Check for fruit characteristics and as the original source of seed to irradiate. NR means data not recorded.

TABLE 2
Leaf characteristics of typical leaves from ‘Tift2-16’ trees and from an
‘Ichang’ tree that were harvested in 2016 at Tifton, GA.

| | Leaf Width | Leaf Length | Leaf Area |
|---|---|---|---|
| Entry | mm | mm | sq cm |
| ‘Tift 2-16’ | 55.3 | 135.3 | 42.9 |
| ‘Ichang’ | 52.3. | 124.4 | 37.6 |
| LSD-5% | 4.5 | 11.2 | 7.2 |

Leaves were measured during November, 2016. Ten leaves were measured from four ‘Tift2-16’ trees. The values are mean values of the measurements.

TABLE 3
Summary of observed morphological characteristics of
‘Tift2-16’ and ‘Ichang’ trees.

| Trait | ‘Tift2-16’ | | ‘Ichang’ | |
|---|---|---|---|---|
| Fruit Circumference | 23.7-30.0 | cm | 29-32 | cm |
| Fruit Weight | 179-372 | g | 343-479 | g |
| Brix | 7.5-12.2 | | 8.6-8.7 | |
| Juice Volume per Fruit | 75-166 | ml | 126-162 | ml |
| Number Seeds per Fruit | 1-8 | | 56 | |
| Mean Leaf width | 55.3 | mm | 52.3 | mm |
| Mean Leaf length | 135.3 | cm | 124.4 | cm |
| Mean Leaf Area | 42.9 | cm² | 37.6 | cm² |

### BRIEF DESCRIPTION OF THE FIGURES

The accompanying colored photographs illustrate the overall appearance and distinct characteristics of the new cultivar of Citrus ‘Tift2-16’. The colors in the photographs are as close as possible with the photographic and printing technology utilized. The photographs are of four year old trees (measured from the time of grafting).

Certain characteristics of this variety, such as growth and color, may change with changing environmental conditions (e.g., light, temperature, moisture, nutrients availability, or other factors).

FIG. 1 is a photograph of a tree with ‘Tift2-16’ scion grafted onto a Poncirus trifoliata rootstock.

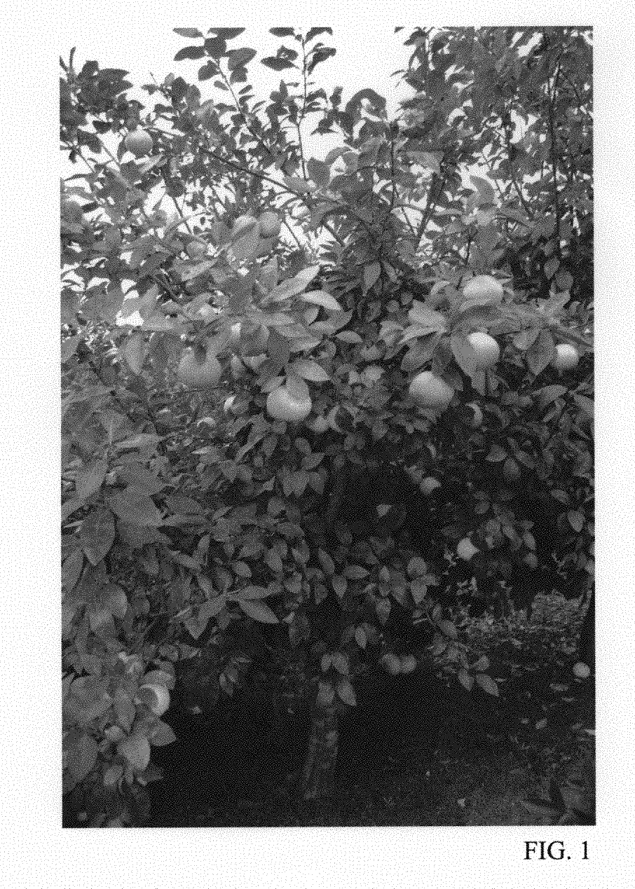

FIG. 2 is a photograph of whole ‘Tift2-16’ fruit attached to a tree.

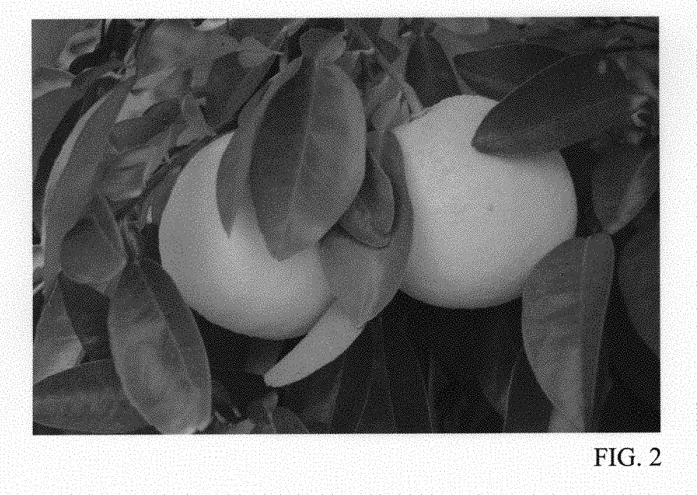

FIG. 3 is a photograph of ‘Tift2-16’ fruit cut in half.

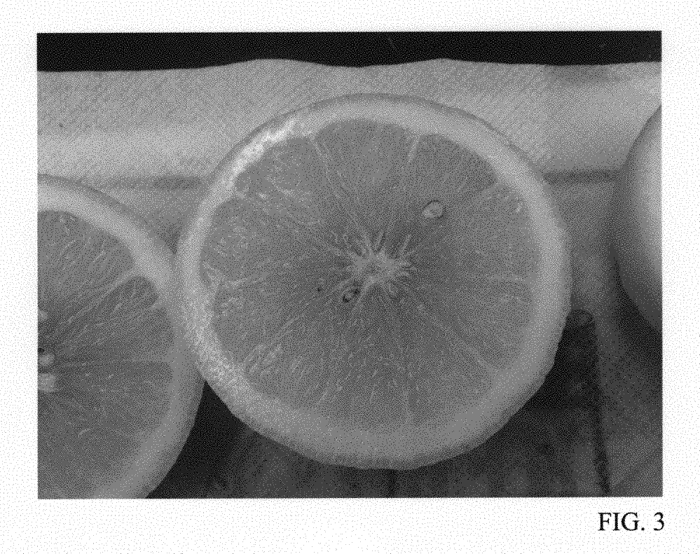

### DETAILED BOTANICAL DESCRIPTION

The described plants of the new cultivar were approximately four years of age, had been asexually reproduced by grafting on Poncirus trifolata rootstock (non-patented) and were observed growing outdoors in full sunlight in Tifton, Ga. Observations obtained during the growing season of 2016 are described below. All colors listed below are with reference to The Royal Horticulture Society (R.H.S.) Colour Chart (Fifth Edition, published 2007).
Classification: Citrus ichangensis x C. maxima.
Tree:
   Vigor.—High.
   Growth habit.—Sparse and spreading. Four year old trees of the new variety growing on Poncirus trifolata rootstock had a height of 265-340 cm and a width or spread of 180-245 cm.
   Bark.—New bark has a smooth texture and has a color of Yellow-Green Group RHS 147C; older bark has a slight texture and has a color of Green-Brown Group RHS 199C.
   Spines.—Moderate number of spines range from none on some limbs, to an average length of 18 mm on some limbs to an average of 33 mm on other limbs and does not appear to be related to age of wood. Spine density is medium on new growth (Green Group RHS N137D) and sparse on mature growth (Green Group RHS N137A).
Foliage:
   Leaf shape. —Generally ovate to elliptical.
   Leaf apex.—Broadly acute.
   Leaf base.—Substantially acute.
   Leaf size.—Relatively long and broad, commonly approximately 135 mm in length (mean value) and 55 mm in width (mean value), and commonly intermediate in thickness.
   Leaf margin.—Mildly crenate.
   Surface texture.—Upper and lower leaf surfaces both smooth with nerve texture.
   Leaf color.—Commonly medium green, commonly near Green Group 137A on the upper surface and near Green Group 137C the lower surface, with an absence of anthocyanin.
   Petioles.—Commonly approximately 29 mm in length and 18 mm in width on average with the presence of wings; color is commonly near Green Group 137A; surface texture is generally smooth.
Inflorescence:
   Habit.—Commonly flowers once per season.
   Self incompatibility.—Absent.
   Fruit parthenocarpy.—Absent.
Fruit:
   Configuration.—Generally round, somewhat flattened at stalk end. Greatest diameter is in the middle region (as illustrated); a depression is present at the stalk end of the fruit.
   Size.—Commonly approximately 23.7-30 cm in circumference and weight 179 to 372 g (mean values). Mean fruit height is 7.8 cm, range 7.3-8.1 cm.
   Neck.—Absent.
   Proximal (stalk) end.—Slightly flattened.
   Distal end.—Truncated.
   Nipple.—Commonly absent.
   Persistence of style.—Commonly none.
   Oil glands.—Small (less than 1 mm).
   Rind.—Commonly thick with medium oiliness.
   Fruit rind adherence to flesh.—Medium.
   Surface.—Glossiness — medium; texture — smooth, color. — commonly near Green-Yellow Group 1D.
   Vesicles.—Length 59.6 mm (Range=55-65 mm); width 21.5 mm (Range 18-25 mm); diameter at surface opposite fruit center 15.3 mm (Range 11-20 mm).
   Albedo.—Color — White Group RHS 155C; dense; large amount adhering to flesh.
   Flesh color.—Commonly near Greyed-Yellow Group 160D.
   Filling of interior.—Very dense commonly with a moderate number of developed segments and the absence of rudimentary segments. Average number of fruit segments 10.6; range 10-11. Fruit core diameter small.
   Seeds.—Commonly approximately 5 to 8 per fruit on average; average seed length is 14 mm (Range 11-17); average width is 9.7 mm (Range=8-13); seed surface texture is smooth; outer seed coat is Grey-Brown Group 199D to White Group 155D; seed shape is spheroid.
   Juiciness.—High juiciness; 75-166 ml per fruit (mean values based on limited sampling); acidity of juice is medium.
   Brix.—7.5-12.2 (mean values based on limited sampling).
   Maturity.—Commonly approximately 210 days to maturity on average in Tifton, Ga.
   Harvest time.—Commonly from about the end of October until about the end of December in Tifton, Ga.
   Market.—Fresh for consumption, and for use in landscaping/backyard plantings.
   Plant disease/pest resitance.—Relatively disease free in areas of testing; mainly have to control leaf miners (Phyllocnistis citrella); rust mites (Phyllocoptruta oleviora); and aphids in general (Aphis sp.)

What is claimed is:

1. A new and distinct cultivar of the Citrus plant named ‘Tift2-16’, as herein illustrated and described.
